(19) United States
(12) Patent Application Publication
(10) Pub. No.: US 2006/0136010 A1
(43) Pub. Date: Jun. 22, 2006

(54) Strial hearing loss treatment device having a sliding electrode

(76) Inventors: Francis A. Spelman, Lake Forest Park, WA (US); Timothy J. Johnson, Kent, WA (US)

Correspondence Address: TIMOTHY E SIEGEL, 1868 KNAPPS ALLEY, SUITE 206, WEST LINN, OR, 97068, US

(21) Appl. No.: 10/918,237

(22) Filed: Aug. 13, 2004

Related U.S. Application Data
(63) Continuation-in-part of application No. 10/780,544, filed on Feb. 17, 2004.
Application No. 10/780,544 is a division of application No. 10/287,989, filed on Nov. 5, 2002, now Pat. No. 6,694,190.
(60) Provisional application No. 60/496,298, filed on Aug. 19, 2003.

(51) Int. Cl. A61N 1/18

(52) U.S. Cl. 607/57

(57) ABSTRACT

An implanted electrolytic current injection device, comprising a reservoir of KCl in electrolytic contact with the interior of the scala media and including a charge injection electrode and a reservoir of saline solution in electrolytic contact with a part of the body that is saline. Also, a current source supplies current to a support electrode, which is moveable between the reservoir of KCl and the reservoir of saline solution. Accordingly, the support electrode may be alternately placed in the reservoir of KCl, for refreshing the charge injection electrode, and in the saline solution, for providing a source of electrons for driving the charge injection electrode. A driver moves the support electrode between the reservoirs.

### RELATED APPLICATIONS

The present patent application claims priority from U.S. provisional application No. 60/496,298 filed Aug. 19, 2003, and from U.S. application Ser. No. 10/780,544 filed Feb. 17, 2004, which is a divisional of U.S. application Ser. No. 10/287,989 filed Nov. 5, 2002, now U.S. Pat. No. 6,694,190.

### STATEMENT OF GOVERNMENT SUPPORT

The invention was made with government support under grant numbers R43DC005531-01 ZRG01 and 2R44DC005531-02. The government has certain rights in the invention.

### FIELD OF THE INVENTION

The present invention is generally related to devices and methods for correcting hearing loss.

### BACKGROUND OF THE INVENTION

As many as seven million Americans suffer from a form of hearing loss known as strial presbycusis, which is marked by a loss of hearing in all registers and, as the name indicates, is associated with the aging process. In a healthy ear there is a voltage difference across the basilar membrane, the organ that hosts the hair cells. This voltage difference, referred to as “endocochlear potential,” causes current to flow through the hair cells. Sound waves cause the hair cells to bend, thereby changing their electrical conductivity and the amount of current that flows through them. This process results in the electrical nerve impulses that are sent to the brain by the auditory nerve.

It appears that the most frequent immediate cause of strial presbycusis is the deterioration of the stria vascularis, a structure that extends along the basilar membrane and produces the ions that create the endocochlear potential. The loss of endocochlear potential appears to result in both an immediate decline in hearing acuity and a gradual deterioration of the structure of the scala media. One potential method of restoring the enodocochlear potential is to inject additional charge by means of an electrode. This is difficult, however, because it requires the production of a DC current within the body. The body's interstitial fluid tends to foul and eventually destroy any implanted electrode producing a DC current. Further, metal electrodes either dissolve or become fouled with new material when they are driven with DC currents.

Because of the tendency for DC electrodes to be fouled, existing therapeutic devices which produce electrical currents within the body, including pacemakers and neural stimulation systems, are driven by charge balanced, biphasic electrical pulses.

### SUMMARY OF THE INVENTION

In a first separate aspect, the present invention is an implanted electrolytic current injection device, comprising a reservoir of KCl in electrolytic contact with the interior of the scala media and including a charge injection electrode and a reservoir of saline solution in electrolytic contact with a part of the body that is saline. Also, a current source supplies current to a support electrode, which is moveable between the reservoir of KCl and the reservoir of saline solution. Accordingly, the support electrode may be alternatingly placed in the reservoir of KCl, for refreshing the charge injection electrode, and in the saline solution, for providing a source of electrons for driving the charge injection electrode. A driver moves the support electrode between the reservoirs.

In a second separate aspect, the present invention is an electrolytic current injection device, implanted in a living body and comprising a reservoir of KCl controllably in electrolytic contact with the interior of the scala media and including an active electrode, the reservoir of KCL also being controllably in electrical contact with a saline portion of the body by way of a structure that does not permit a harmful level of ion transport between the KCl reservoir and the saline portion of the body. Also, a reservoir of saline solution is in electrolytic contact with a part of the body that is saline and including a refresh electrode. Additionally, a current source is electrically interposed between the active electrode and the refresh electrode. A controller places the current injection device into a current injection mode in which the current source creates electric current flow from the refresh electrode to the active electrode and simultaneously places the KCl reservoir into electrolytic contact to the scala media, thereby causing charge to be electrolytically injected into the scala media. Alternately, the controller places the current injection device into a refresh mode in which electric current flows from the active electrode to the refresh electrode and the KCl reservoir is removed from electrolytic contact to the scala media and into electrical contact to the NaCl portion of the body, thereby causing a refreshing electrolytic current into the refresh electrode.

The foregoing and other objectives, features and advantages of the invention will be more readily understood upon consideration of the following detailed description of the invention, taken in conjunction with the accompanying drawings.

### BRIEF DESCRIPTION OF THE DRAWINGS

FIG. 1 is an illustration of an implantable charge injection assembly and driver, according to the present invention, shown implanted in the skull.

FIG. 2 is an illustration of the implantable charge injection assembly and driver of FIG. 1, shown in relation to the structure of the inner ear.

FIG. 3 is an illustration of the implantable charge injection assembly of FIG. 1, shown in greater detail.

FIG. 4 is a greatly expanded illustration of an electrostatically actuated micro machined gate, in its closed state, as utilized in the present invention.

FIG. 5 is a greatly expanded illustration of an electrostatically actuated micro machined gate in its open state, as utilized in the present invention.

FIG. 6 is an illustration of an alternative embodiment of an implantable charge injection assembly, which includes membranes that controllably and selectively permit the passage of electrolytes.

FIG. 7 is an illustration of an additional alternative embodiment of an implantable charge injection assembly, which uses electromagnetic current steering.

FIG. 8 is an illustration of an additional alternative embodiment of an implantable charge injection assembly, which has a rotatable electrode.

FIG. 9 is an illustration of an additional alternative embodiment of an implantable charge injection assembly, which has two charge injection units.

FIG. 10 is an illustration of an additional alternative embodiment of an implantable charge injection assembly, which has two charge injection units, but having a different construction from that of FIG. 9.

FIG. 11 is a timing diagram for the assembly of FIG. 9, but that would apply equally as well (with analogous labeling) to the embodiment of FIG. 10, and the embodiment of FIGS. 12 and 13.

FIG. 12 is a schematic diagram of an additional alternative embodiment of an implantable charge injection assembly, showing the assembly in a first state.

FIG. 13 is a schematic diagram of an additional alternative embodiment of an implantable charge injection assembly, showing the assembly in a second state.

FIG. 14 is a schematic diagram of yet another alternative embodiment of an implantable charge injection assembly.

FIG. 15A is a schematic diagram of a half wave rectification charge injection device according to the present invention, in charge injection mode.

FIG. 15B is a schematic diagram of a half wave rectification charge injection device according to the present invention, in electrode refresh mode.

FIG. 16A is a schematic diagram of an alternative embodiment of a half wave rectification charge injection device according to the present invention, in charge injection mode.

FIG. 16B is a schematic diagram of the charge injection device of claim 16A, in active electrode refresh mode.

### DETAILED DESCRIPTION OF THE PREFERRED EMBODIMENTS

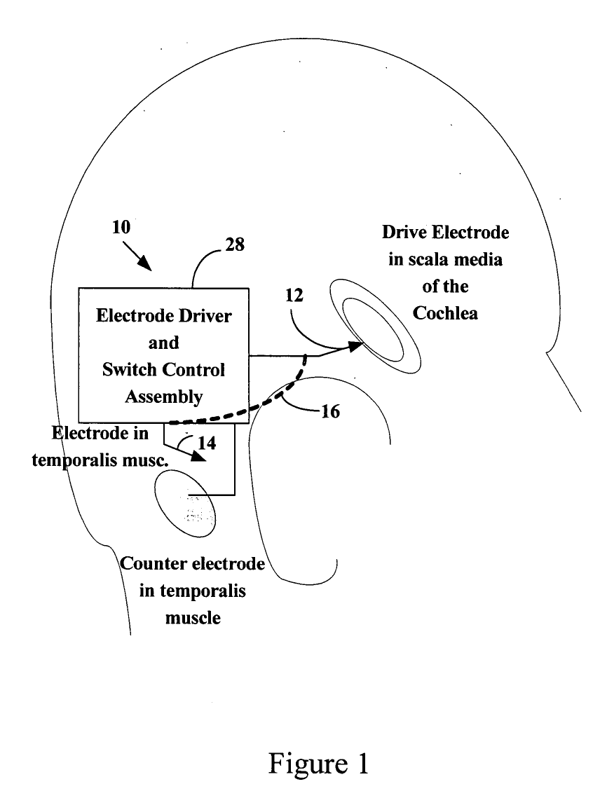

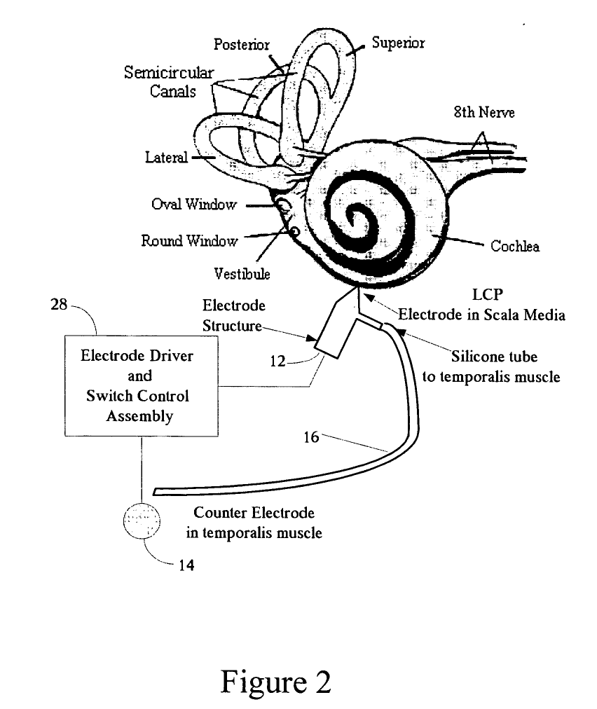

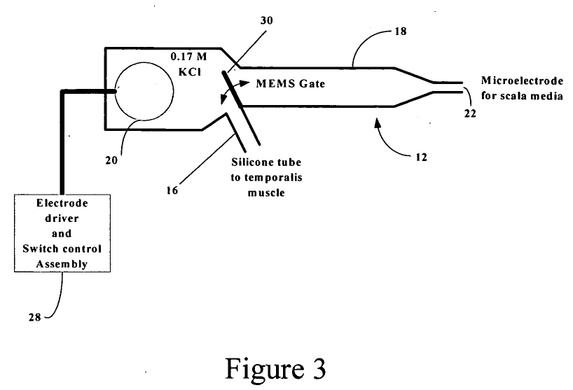

Referring to FIGS. 1 and 2, an implantable charge injection assembly 10 according to the present invention, is designed to be implanted in the human skull. A charge injection unit 12 will be placed so that it contacts the scala media of the subject. In one preferred embodiment, the structure of charge injection unit 12 includes an electrolytic fluid-filled liquid crystal polymer (LCP) housing 18 (FIG. 3). The electrolytic fluid is an aqueous solution of _{—}0.17_M KCl to match the potassium concentration of human scala media tissue. Referring to FIG. 3, a primary electrode 20 located in the housing 18 is made of conductive metal plated with IrOx and has a surface area of 1.6×10⁹ μm². Injection unit 12 includes a tip 22 that contacts the scala media and has an interior area that is less than one hundred thousandth that of electrode 20, being between 100 μm² and 10,000 μm². The length of the tip 22 is 0.2 mm to 0.5 mm.

The dimensions of charge injection unit 12 determine the bulk of the DC resistance of unit 12, which equals about 0.1 to 1 megohms, based on a resistivity of 36.7 ohm-cm for 0.17 M KCl at 37° C.

Charge injection assembly 10 includes a tube 16 that extends from unit 12 to a refresh electrode 14 that is embedded in the temporalis muscle, or that may be located in a closed side chamber of the electrode assembly. Tube 16 has an inside diameter of 25 μm or more and is filled with KCl liquid of appropriate molarity.

An electrode driver and switch control assembly 28 controls a micro machined gate 30 assembly with flap 32(FIGS. 3 4 and 5), which exposes electrode 20 to either tip 22 or refresh electrode 14. When the gate assembly 30 is positioned to connect electrode 20 to tip 22, assembly 28 drives electrode 20 to cause it to inject charge into the scala media by way of tip 12. When the gate assembly 30 is positioned to connect electrode 20 to the refresh electrode 14, electrodes 20 and 14 will be driven so that electrolytic current flows into and thereby refreshes primary electrode 20, analogous to half-wave rectification. The single bi-state gate could also be replaced by two separate single-state gates operating in opposite phase from one another.

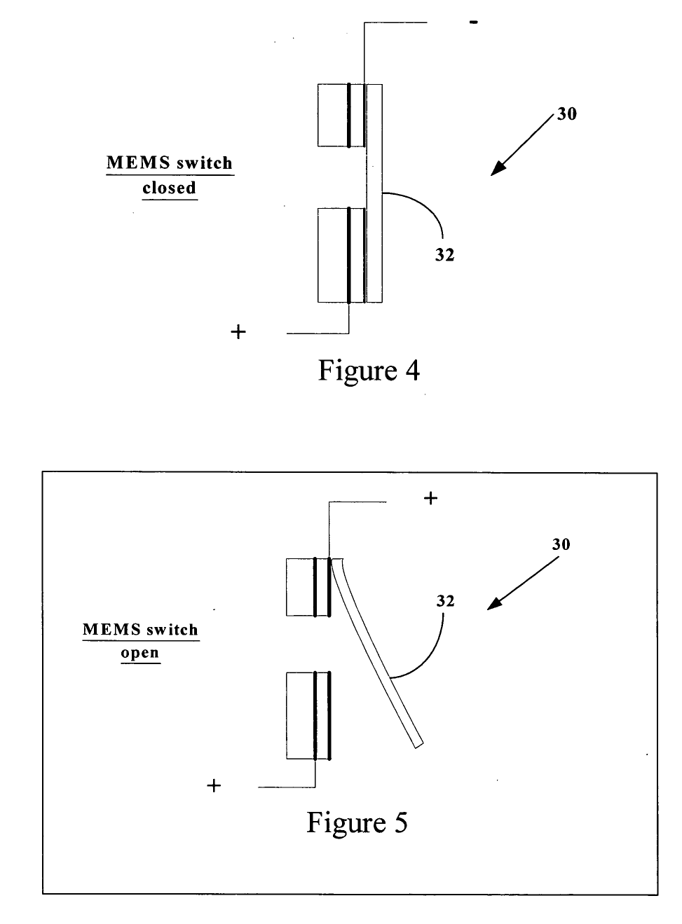

Referring to FIGS. 4 and 5, in one preferred embodiment gate 30 is electrostatically actuated. Gate 30 is made by the photolithographic conductive structures on thin sheets of liquid crystal polymer (LCP) combined with the laser micromachining of a small flap 32. The flap 32 is kept closed by maintaining a small opposite charge on electrodes placed on the surfaces of flap 32. The facing electrodes are electrically separated by a surface dielectric. To open the switch, like polarity is applied to both electrodes. By utilizing LCP material, which is thermoplastic, material can be selectively adhered by spot “welding” using an IR laser, or selectively removed using a UV laser, allowing a variety of designs to be implemented. In an alternative approach, the gate is mechanically pre-biased to remain closed. The bias is then overcome electrostatically to actuate the gate.

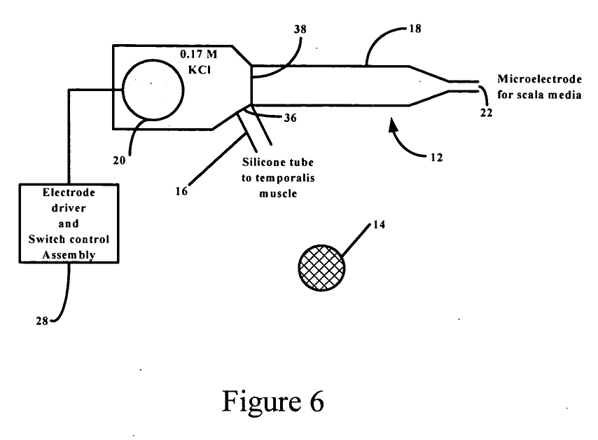

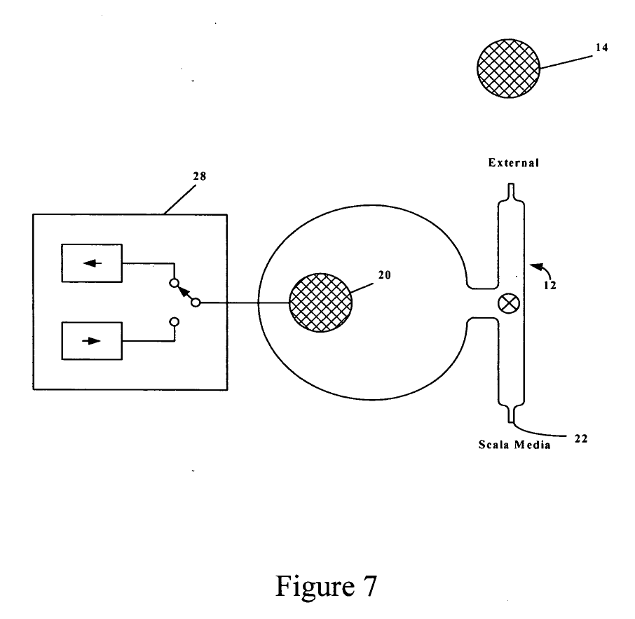

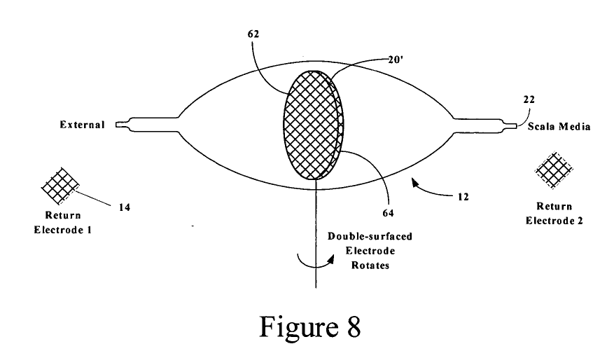

Referring to FIG. 6, in an alternative preferred embodiment, a pair of ion-selective membranes 36 and 38 that permit the flow of positive ions from electrode surface 20 in a direction toward the tip of the electrode 22, while simultaneously allowing the flow of negative ions from electrode 14 and surrounding tissue. In an additional alternative preferred embodiment, shown in FIG. 7, a magnet steers the electrolytic current to selectively connect electrode 20 with electrode 14 or tip 22. When the electrolytic current changes its direction from the electrode, it is steered by the magnetic field so that positive current flows into the scala media and negative current flows to the refresh electrode. The interaction of DC currents with DC magnetic fields causes this effect. In yet another preferred embodiment, shown in FIG. 8, a primary electrode 20′ is rotatable, so that a first face 62 can be refreshed while a second face 64 is actively injecting current into the scala media.

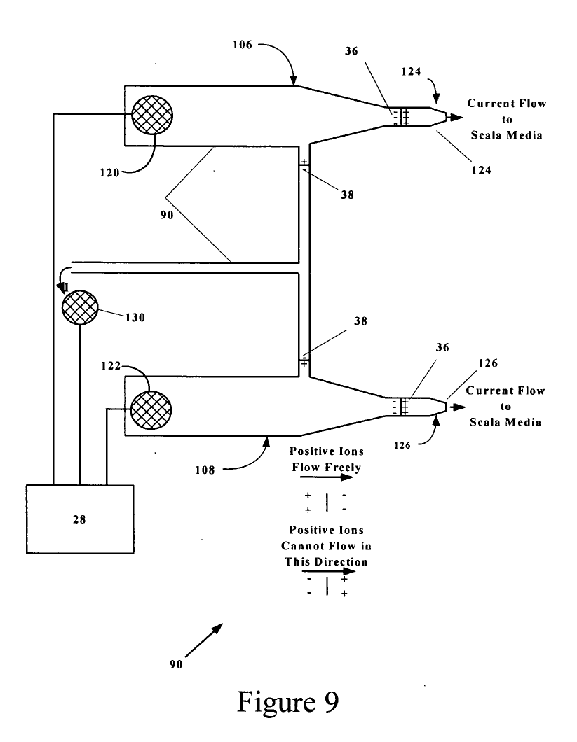

Electrode 20 (or 20′) is capable of passing a current of 10 μA for a duration of 3-6 sec through tip 22 and into the scala media. Scientific investigation has indicated that during the 3-6 second refresh periods for electrode 20, the potential across the basilar membrane will persist. Referring to FIG. 9, an additional preferred embodiment of a charge injection assembly 90 permits a continuous injection of charge into the scala media, analogous to full-wave rectification. Patients that have a damaged scala media, which is less capable of storing charge, may prefer this embodiment. Assembly 90 includes a pair of charge injection units 106 and 108, which are toggled in their active states by an electrode driver and switch control assembly 28 controlling ion selective membranes 36 and 38 to maintain a continuous charge injection. Units 106 and 108 include a pair of driving electrodes 120 and 122 respectively, and a pair of tips 124 and 126 respectively. One or more refresh electrodes 130 are used to maintain electrodes 120 and 122, so that an injection of charge into the scala media can be continuously maintained, by switching between tips 124 and 126. In an alternative embodiment, the duty factor of the charge injection is increased, but is still not continuous.

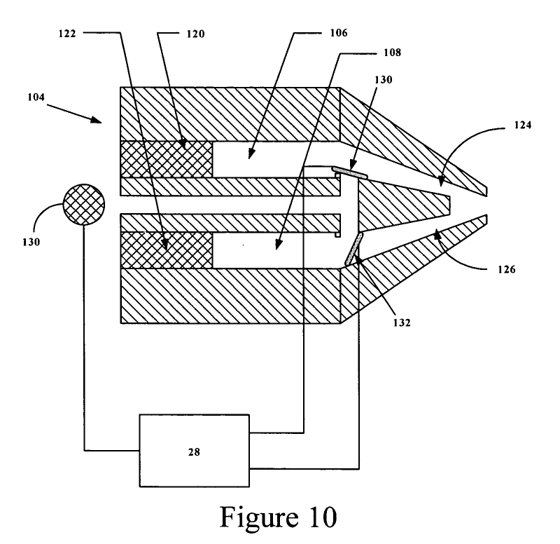

Referring to FIG. 10, an alternative embodiment of an assembly 104 is conceptually the same as assembly 90 except for that instead of ion selective membranes 36 and 38 a pair of MEMS switches 130 and 132 are used for alternately occluding unit 106 and 108.

For any of the above described embodiments, the current driver and switch control assembly 28 is sized to drive a maximum current of 5-30 μA in either direction. In one preferred embodiment, in which the resistance of unit 12 is 1 MΩ, the driver is designed to remain linear over a range of at least ±30 volts. In another preferred embodiment, the dimensions of unit 12 are altered so as to reduce the resistance of unit 12. In another preferred embodiment the voltage level of the fluid of the scala media is measured and used to regulate the amount of current injected. It is noted that a large peak voltage has the potential for causing damage to body tissue and should generally be avoided.

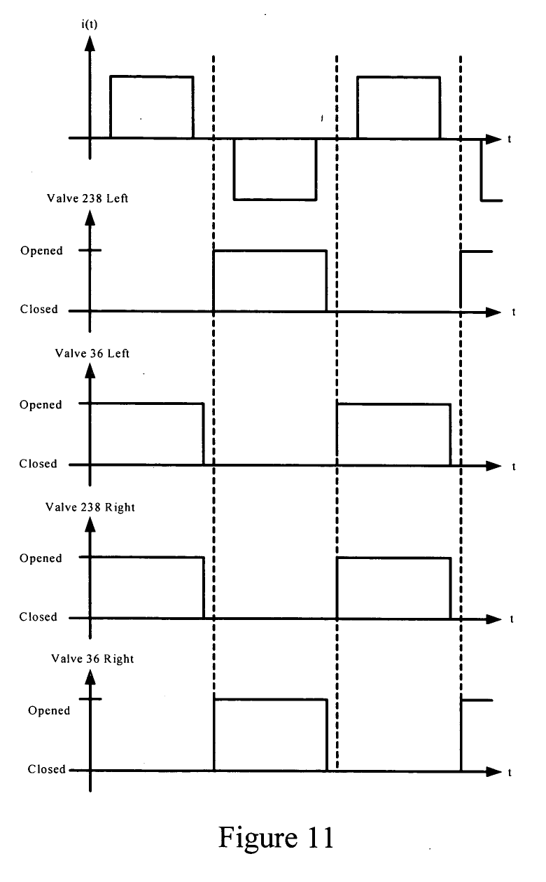

FIG. 11 shows the logic of assemblies 90, 104 and 210 (see below), where i(t) is the current applied from the current generator, and the other graphs in the sketch of the logic show the positions of the MEMS switches. Note that the current drive is discontinuous and that the time that the drive is applied during each half cycle is less than the total time of a half cycle. Current is delayed at the beginning of each half cycle to ensure that the MEMS gates are properly opened and closed before current flows through the system. Current is shut off prior to the end of each half cycle to ensure that no current will be driven during the time that the MEMS gates close. In summary, while current is unidirectional (injected) into the scala media, it is not true DC, but is interrupted.

One problem encountered with the use of the systems described above is that they may permit sodium ions from the body tissue outside the scala media to corrupt the scala media fluid, which is rich in potassium ions. Likewise, potassium ions from the scala media may migrate into and damage body tissue.

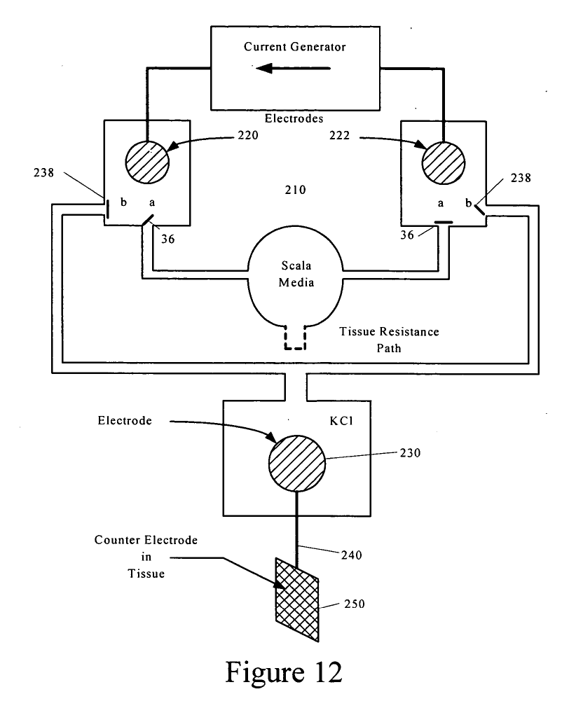

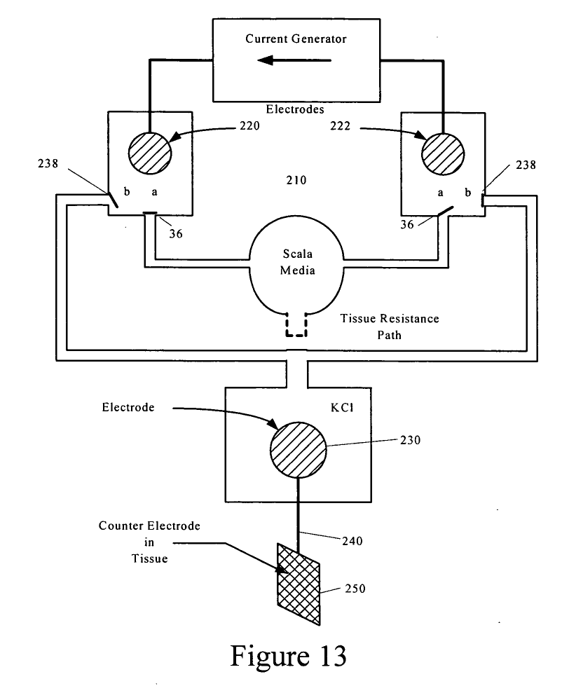

FIGS. 12 and 13 show a charge injection assembly 210 designed to overcome the problem that is outlined in the paragraph above. The assembly 210 is modified to be fully closed and isolated from the tissue, save through a pair of valves 236 leading into the scala media. KCl is confined to the assembly 210 and to the scala media, where it is found naturally. A third metallic electrode 230 is contained in the KCl-filled electrode assembly. That third electrode is connected by a metallic conductor 240 to a fourth electrode 250, which is embedded in the sodium-rich tissues that are external to the scala media via a fourth. This design contains the potassium-rich solutions in tissues where potassium is the normally the dominant ion. It provides a return path for the two active electrodes 220 and 222, by way of valves 238.

FIG. 12 shows the implementation of assembly 210 with current flowing from electrode 220, via the scala media and external tissue, through the external electrode 230 and thence to the right-hand assembly electrode 222, which is negatively charged. FIG. 13 reverses the process.

Since current is not driven with a 100% duty cycle, as is described in the text associated with FIG. 11. The absence of current for a portion of the time, permits the internal electrode 230 and external electrode 250 to depolarize relative to each other.

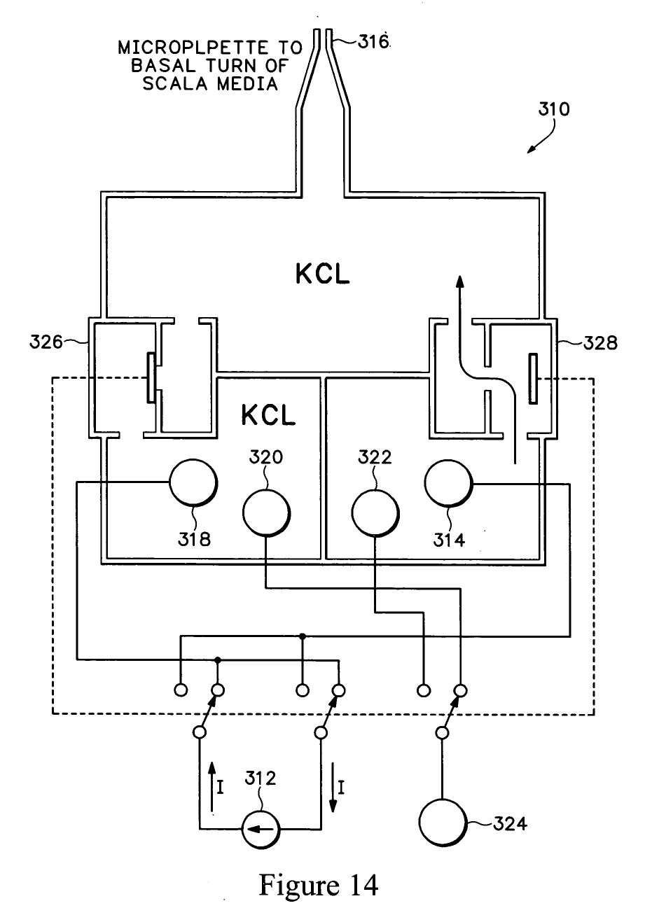

An alternative embodiment is shown in FIG. 14. As shown, current source 312 is injecting current into the scala media by way of electrode 314 and micropipette 316. At the same time, electrode 318 is being refreshed by drawing electrolytic current in from an electrode 320, which is electrically connected to a temporalis muscle-implanted electrode 324. Alternating with the phase shown is a phase in which all of the switches are moved to their other polarities, electrode 314 is refreshed by electrolytic current originating at electrode 322 and electrode 318 injects current into the scala media. MEMS valves 326 and 328 are alternatively opened and closed, placing electrode 312 and then electrode 318 into electrolytic contact with the scala media in alternating sequence.

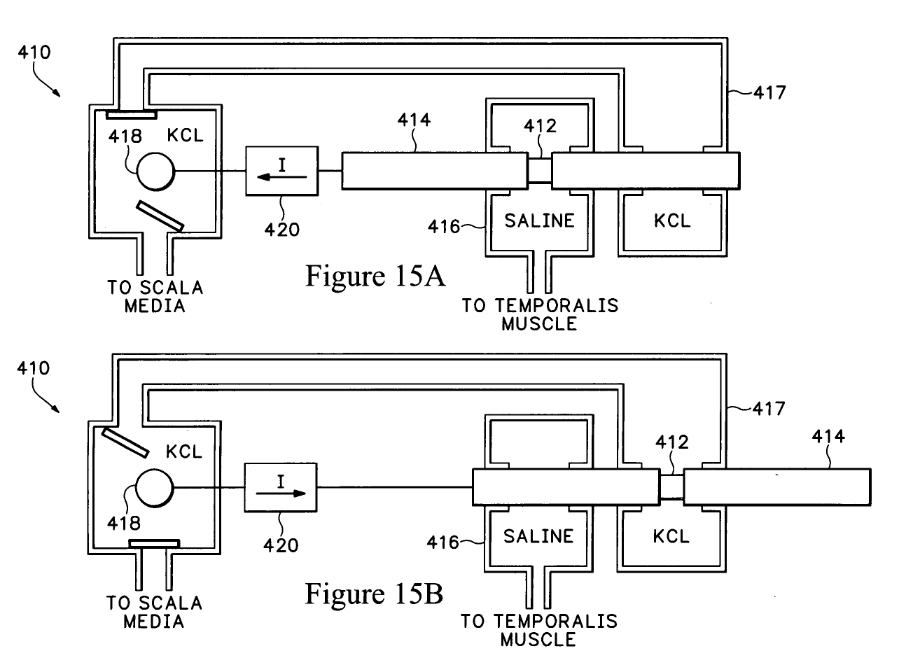

FIGS. 15A and 15B show a half wave rectifying charge injector 410, in which an electrode 412 placed on a slidable boom 414 is slid into a reservoir 416 of saline solution in order to drive a charge injector electrode 418. On alternating phases, electrode 412 is slid into a reservoir of KCl that is in fluid communication with charge injector electrode 418, for the purpose of refreshing electrode 418. During both phases, current source 420 drives electrodes 412 and 418. Boom 414 may be moved by a nitinol wire, cilliary actuator arrays or gas actuation using either heated gases or electrolytically generated gases.

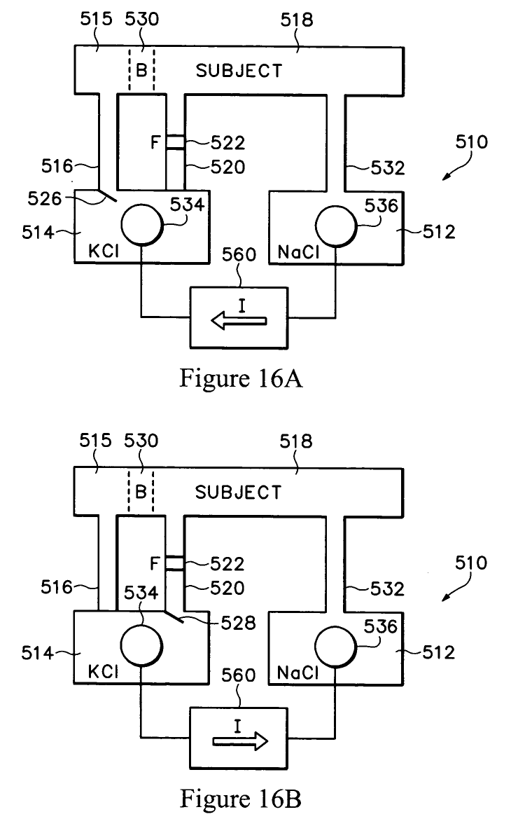

Referring to FIGS. 16a and 16b, an alternative preferred embodiment of a current injection device 510, similar to the embodiment of FIGS. 15a and 15b, has a NaCl reservoir 512 and a KCl reservoir 514. The KCl reservoir 514 is connected to the scala media 515 by a passageway 516 also filled with water bearing KCl ions. Passageway 516 is selectively closeable by way of a valve 526. The KCl reservoir is also electrically connected to NaCl bearing body tissue 518 by way of a passageway 520 filled with water bearing KCl ions, but that is blocked to fluid movement by way of a frit 522, which is electrically conductive. In an alternative embodiment, passageway 520 is so long and thin as to prevent a harmful level of ion transfer.

A valve 528 controls the electrolytic connection between KCl reservoir 514 and passageway 520. A natural barrier 530 of body tissue prevents any harmful level of ion transfer between NaCl bearing tissue 518 and the KCl fluid fed in NaCl reservoir 512. A current source 540 may be controlled to create current from refresh electrode 536 to active electrode 534 or vice versa.

A controller (not shown) either places device 510 into a current injection mode (FIG. 16A), in which current is injected into the scala media or an active electrode refresh mode. In injection mode, the current source 560 sends electric current from refresh electrode 536 to the active electrode 534. The circuit is completed by opening valve 526 thereby placing KCl reservoir 514 into contact with the scala media. Consequently the electric current flow from refresh electrode 536 to active electrode 534 is balanced by electrolytic current flows from KCl reservoir 514 to the scala media 515 and from NaCl tissue 518 to NaCl reservoir 512. The circuit is completed by a movement of electrical charge through barrier 530, which is somewhat electrically conductive.

In refresh mode the current source 560 is reversed so that electric current flows from active electrode 534 to refresh electrode 536. In this mode, also, valve 526 is closed and valve 528 is opened so that electrolytic current flows from glass frit 522 to active electrode 534, thereby refreshing electrode 534. Electrolytic current flows from NaCl reservoir 512 to NaCl tissue 518 and through a portion of passageway 522 to glass frit 522. Electric current passes through glass frit 522, completing the circuit.

An alternative preferred embodiment is schematically very similar to the embodiment of FIG. 6 but without tube 16 or valve 36, and having two further innovations. First, the active electrode 20 and the counter or refresh electrode 16 are both expanded in surface area, to have a surface area of greater than 1 cm² and in one preferred embodiment in the range 10-100 cm² or greater. This can be accomplished using technology similar to that employed in the production of batteries and/or capacitors, in which foil is wrapped about itself or a set of conductive plates are joined together in close proximity to one another.

In this alternative embodiment, also, the frequency of charge injection and refresh could be greatly slowed down, with the object of starting to inject charge slightly before the patient awakens and for the subsequent ten hours, so that during the waking day the patient has a proper voltage gradient across the hair cells. Then, at night time the refresh cycle could occur, when the patient is not in as great need of keen hearing. For this to work properly it is desirable to form electrodes 14 and 20 from a material that has a high (>25 mC/cm²) charge storage capacity, such as iridium oxide film, known in the industry as “IROF.”

The terms and expressions which have been employed in the foregoing specification are used as terms of description and not of limitation, and there is no intention, in the use of such terms and expressions, of excluding equivalents of the features shown and described or portions thereof, it being recognized that the scope of the invention is defined and limited only by the claims which follow.

1. An implanted current injection device, comprising:
(a) a first reservoir of a first electrolyte in contact with the interior of the scala media and including a charge injection electrode;
(b) a second reservoir of a second electrolyte in contact with a part of the body that is external to the scala media;
(c) a current source; and
(d) a support electrode that is electrically connected to said current source, said support electrode being moveable between said first reservoir and said second reservoir so that said support electrode may be alternatingly placed in said first electolyte, for injecting current into scala media, and in said second electrolyte, for refreshing said charge injection electrode; and
(e) a driver for moving said support electrode between reservoirs.

2. An electrolytic current injection device, implanted in a living body and comprising:
(a) a first reservoir of electrolyte controllably in electrolytic contact with the interior of the scala media and including an active electrode, said reservoir of electrolyte also being controllably in electrical contact with a portion of said body external to scala media by way of a structure that does not permit a harmful level of ion transport between scala media and portion of said body external to scala media;
(b) a second reservoir of electrolyte in contact with a part of the body that is external to scale media and including a refresh electrode;
(c) a current source electrically interposed between said active electrode and said refresh electrode; and
(d) a controller adapted to place said current injection device into a current injection mode in which said current source creates electric current flow from said refresh electrode to said active electrode and simultaneously places said first reservoir into electrolytic contact to said scala media, thereby causing charge to be electrolytically injected into said scala media and adapted to alternately place said current injection device into a refresh mode in which said current source creates electric current flow from said active electrode to said refresh electrode and said first reservoir is removed from electrolytic contact to said scala media and into electrical contact to said portion of said body external to scala media, thereby causing a refreshing electrolytic current into said refresh electrode.
